(19) United States
(12) Patent Application Publication
(10) Pub. No.: US 2011/0183410 A1
(43) Pub. Date: Jul. 28, 2011

(54) BIOMASS POWER PLANT

(76) Inventor: Rudolf Loock, Hamburg (DE)

(21) Appl. No.: 13/056,084

(22) PCT Filed: Dec. 22, 2008
(86) PCT No.: PCT/EP08/11033

§ 371 Date: Apr. 7, 2011

(51) Int. Cl. C12M 1/00; C12M 1/12

(52) U.S. Cl. 435/297.1; 435/289.1

(57) ABSTRACT

The invention relates to a biomass power plant for dry-wet simultaneous fermentation, having dry fermenter modules comprising dry fermenters. In order to refine the biomass power plant such that the length of the pipes for delivering process water is reduced to a minimum, the invention proposes to integrate a process water reservoir between two dry fermenter modules.

### TECHNICAL FIELD

The invention relates to a biomass power plant for dry-wet simultaneous fermentation having dry fermenter modules comprising dry fermenters.

### PRIOR ART

Biomass power plants of the type described in the introduction are known from the prior art and have been developed in particular by the Loock engineering company, Hamburg.

Dry-wet simultaneous fermentation (the Loock-TNS-Verfahren™) was developed as a dry fermentation process for generating biogas from solid biomass. In the related art, this technology is considered to be a proven vehicle for pursuing new approaches in the fermentation of organic substances with high dry substance fractions (DS).

Biomass power plants of the kind described in the introduction essentially consist of two main plant sections in which organic substances are converted into biogas, that is to say the dry fermenter modules which in turn comprise multiple dry fermenters, and a process water reservoir. The cogeneration unit for converting the resulting biogas into electricity is also present.

In a biomass power plant of the kind described in the introduction, the biogas is converted with conditioned process water in the dry fermenters by controlled irrigation of the substrate, the water being circulated, generated and maintained in a regulated circuit. For this, the substrate pile in the respective dry fermenter is subjected to uniform moisture penetration. The process water percolates through the substrate and is then fed to the process water reservoir, which has the form of a gas-impermeable tank.

Organic acids in the substrate are dissolved by hydrolysis and carried to the process water reservoir by means of the percolate, said organic acids serve as feed for microorganisms. Consequently, a methane-forming biology forms inside the closed, gas-tight process water reservoir and is continuously replenished with nutrients by the hydrolysis taking place in the dry fermenters. Biogas is generated continuously in the process water reservoir at the same time as biogas is being produced by fermentation of the substrate with high solid content in the dry fermenters.

The circular flow system of the water has a further advantage in that the microbiology already present in the process water is used to seed fresh substrate in the dry fermenters. In this way, the fermentation process is accelerated and usable biogas is generated in the dry fermenter after a significantly shorter priming period.

This introductory description already shows that the process water reservoir is an important element of the biomass power plant. The part of the biomass power plant that consists of dry fermenters arranged side by side is located at various distances from the process water reservoir depending on the site. Insulated pipes of corresponding lengths must be manufactured for transporting the temperature-controlled process water, which in turn represents a great deal of expenditure in terms of construction and costs.

### DESCRIPTION OF THE INVENTION, OBJECT, SOLUTION, ADVANTAGES

In the context of these disadvantages and taking into account the state of the art as outlined, the object of the present invention is therefore to improve a biomass power plant of the type described in the introduction to such effect that the lengths of the process water pipes between the dry fermenter modules and the process water reservoir are reduced to a minimum.

This object task is solved with the features of claim 1. Advantageous embodiments of the invention will be evident from a reading of the dependent claims.

According to the invention, a process water reservoir is integrated between two dry fermenter modules comprising preferably four dry fermenters each.

The underlying idea of the invention is to locate the process water reservoir as closely as possible to the dry fermenter modules in order to reduce the length of the pipelines for the process water to the minimum possible. Since the dry fermenters in a biomass power plant are arranged modularly, that is to say in a number of blocks, it is provided according to the invention that the process water reservoir is arranged between the dry fermenter modules, so that the pipelines for transporting the process water are able to be arranged to a certain degree symmetrically with each other from the process water reservoir to the dry fermenter modules. In this way, it may be ensured that the length of the pipelines between the dry fermenter modules and the process water reservoir is reduced to a minimum, and that production of the pipelines may be streamlined by virtue of their symmetrical arrangement.

To ensure that the machine technology necessary for operating the biomass power plant is used as efficiently as possible, according to one advantageous embodiment of the invention eight dry fermenters are arranged in two blocks of four, that is to say each dry fermenter module comprises four dry fermenters.

It is advantageous that the dry fermenters have an interior width of no more than 4.5 m and an interior height of no more than 5.0 m. These dimensions have proven to be particularly advantageous, since this enables the desired vertical and horizontal formation of capillaries given the substrate volume that results therefrom.

It is further provided within the scope of the invention that the dry fermenters are equipped with laterally installed lattice segments with gutters to allow the process water to drain away. One practical variant of the invention provides that the lateral lattice segments are made from stainless steel and/or plastic.

It must also be ensured that the substrate in the dry fermenter does not become too acidic due to its own acidogenic potential released in the first hydrolysis phase. The efficiency of the process must be encouraged by enabling the bacteria from the process water to be established (seeding) and begin metabolising quickly. Low pH values inhibit the methane-forming bacteria. This is why the need to provide means for dewatering the dry fermenters effectively by enabling water to drain off laterally from the substrate pile was a further important insight of the invention. In technical terms, this effective dewatering is achieved according to the invention by the lattice segments.

The lattice segments are preferably inclined in such manner that their distance to the side walls of the dry fermenters increases from bottom to top, that is to say the lattice segments are truncated. In particular, an inclination of 2° has proven advantageous. One advantage of the truncated lattice segments is that they enhance the water's ability to flow through the substrate pile and cause less compaction of the substrate pile.

With regard to irrigation of the substrate pile with process water, it must be ensured that water is introduced evenly over the entire surface of the substrate pile to ensure that fermentation takes place uniformly throughout the substrate volume. This is achieved according to the invention for example with a row of nozzles introduced in the fermenter roof, preferably in the form of full cone nozzles. Fibres are also carried out of the substrate together with the water draining from dry fermenters. Since the process water is being circulated continuously, these fibres become concentrated in the process water over time. To prevent the nozzles from becoming clogged by these fibres, a self-cleaning cyclone filter is integrated in the process water pipeline system.

Isobaric air distribution devices are advantageously fitted in the floors of the dry fermenters.

For this purpose, the use of specially developed compressed air distributors in the form of isobaric sword nozzles installed in the centre of recesses in the fermenter floor, and for which the air supply lines pass along one of the long walls of the fermenter and out through the roof has emerged as the most practical solution.

The ideal device is an arrangement of ventilation nozzles via which compressed air may be introduced in short blasts into the substrate pile from below as well as a continuous flow of air generated by a side channel blower.

Atmospheric air is introduced directly into the substrate pile with this combination of compressed and continuous ventilation installed in the floor. This serves advantageously to loosen the substrate pile and aerate it evenly. The defined flow of exhaust air may be collected and fed to an exhaust air treatment system with no additional effort. This combination of ventilation methods in the floor is a highly effective method of carrying out the aeration that must always be included in batch processes to ensure the absence of gas before the dry fermenter is opened and emptied.

The power plant advantageously includes a process water collecting duct that is coupled to the process water reservoir and/or the dry fermenter in the form of a module.

A further practical variant of the invention provides that the process water collecting duct has a volume of at least 50 m³. This ensures that the process water collecting duct is also able to perform the function of a temporary storage volume for the percolate.

The process water collecting duct is preferably arranged opposite a side of the process water reservoir that is not facing the dry fermenters.

A further advantageous embodiment of the invention provides that the machine technology, piping, pumps, blowers and boosters are arranged above the dry fermenter modules and the process water reservoir.

It is advantageous if a gas storage container and/or a biofilter are arranged on the sides of the dry fermenter modules facing away from the process water reservoir.

### BRIEF DESCRIPTION OF THE FIGURES

The invention will be explained in greater detail below with reference to the figures. The diagrammatic figures show:

FIG. 1 a plan view of a biomass power plant according to the invention;

FIG. 2 a cross sectional view of the biomass power plant of FIG. 1 along line

B-B in FIG. 1 and

FIG. 3 a cross sectional view of the biomass power plant of FIG. 1 along line A-A in FIG. 1.

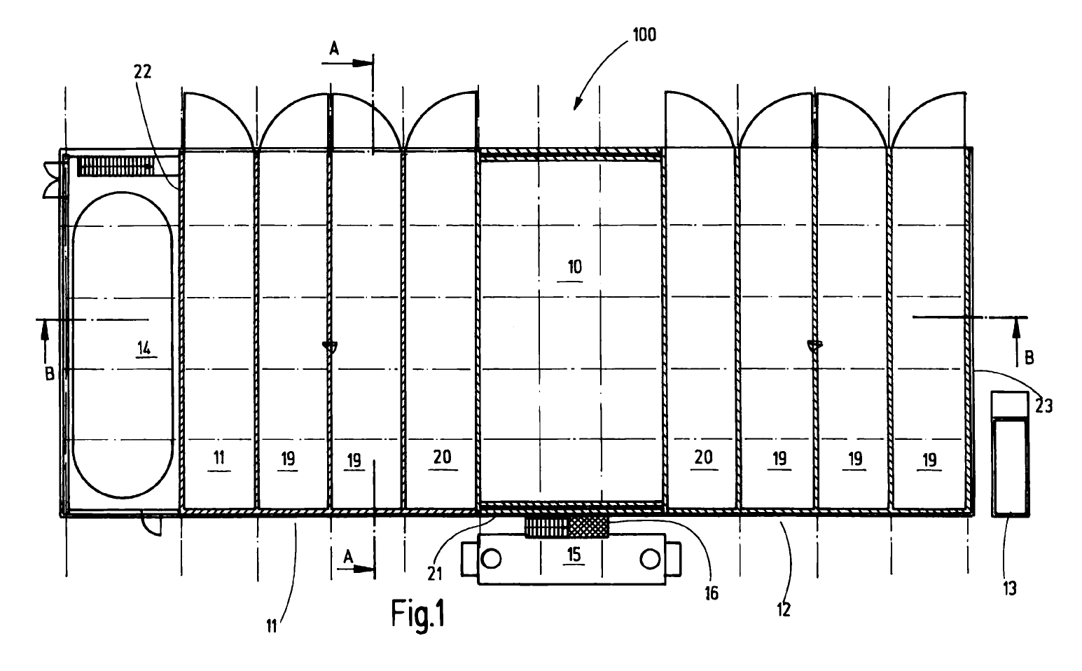

FIG. 1 shows a biomass power plant according to the invention, which is designated by the reference number 100.

### BEST MODE TO REALIZE THE INVENTION

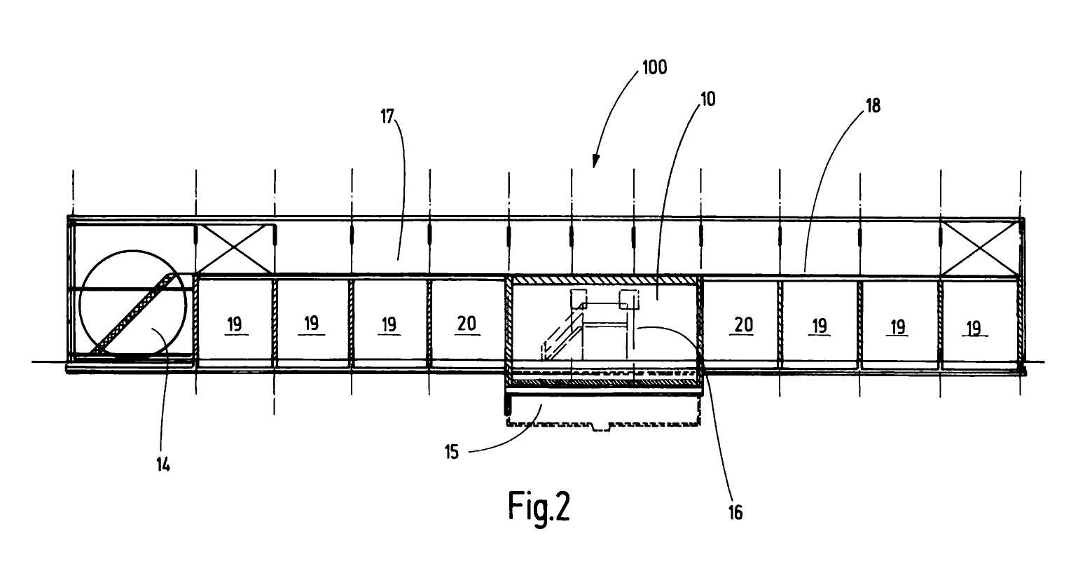

FIG. 1 shows a number of essential components that are necessary for the operation of biomass power plant 100, in particular process water reservoir 10, dry fermenter modules 11, 12 and biofilter 13 as well as gas storage receptacle 14, wherein connection conduits, in the form of pipelines for example, are not shown between these components in FIGS. 1 to 3. These components, which are located in a hall of biomass power plant 100 not shown in FIG. 1, are supplemented by process water collecting duct 15 and staircases 16 that give access to the dry fermenter roofs 17, 18 shown in FIG. 2, where the machine technology, piping, pumps, blowers and some of the pipelines for transporting the process water are located.

Eight dry fermenters 19, 20 having internal dimensions of about 20 m×4 m×4 m are arranged in two blocks of four, that is to say in the form of dry fermenter modules 11, 12 in the hall which is not shown explicitly in FIG. 1. Process water reservoir 10 is integrated between dry fermenter modules 11, 12 in such manner as to ensure that dry fermenter modules 11, 12 and process water reservoir 10 are located as closely as possible to each other. In this way, it is ensured in a manner critical for the invention that the pipelines necessary for transporting the process water, not shown in FIG. 1, between process water reservoir 10 and dry fermenters 19, 20, are reduced to a minimum. This further assures in a manner critical for the invention that the pipelines that extend from process water reservoir 10 to dry fermenters 19, 20 are able to be disposed symmetrically with each other. Staircase 16 affords access to the dry fermenter roofs, where for example the ventilation units, the pumps necessary for irrigating the substrate in the dry fermenters, and other machine technology is located (FIG. 2). Thus, the biomass power plant 100 according to the invention differs from the biomass power plants known from the prior art in that process water reservoir 10 is not spatially separated from dry fermenter modules 11, 12. Instead, dry fermenters 19, 20 are located directly beside one another, and process water reservoir 10 and the two dry fermenters 20 closest to process water reservoir 10 are also arranged directly adjacent to each other. Incidentally, another advantageous result of this compact design is that the throughput times associated with percolation in the regulated circuit are also shortened.

As is also evident in FIG. 1, biomass power plant 100 includes a process water collecting duct 15 that is coupled to process water reservoir 10 and dry fermenters 19, 20 in the form of a module. It is thus provided according to the invention that process water collecting duct 15 is arranged in the immediate vicinity of dry fermenter modules 11, 12 and of process water reservoir 10 as a replaceable component, that is to say in modular manner. Consequently, a process water collecting duct that is embedded and cemented into the ground is not provided according to the invention.

In the embodiment of the invention shown in FIG. 1, process water collecting duct 15 is located facing the side of process water reservoir 10 that is not facing dry fermenters 19, 20. The process water collecting duct has a volume of at least 50 m³, so that process water collecting duct 15 may also perform the function of a temporary storage receptacle for percolate.

As a further technical step, it is provided within the scope of the invention that a gas storage tank 14 and a biofilter 13 are located on the sides 22, 23 of dry fermenter modules 11, 12 facing away from process water reservoir 10. Biofilter 13 serves in known manner to clean the exhaust air from dry fermenters 19, 20 and is also modular in design. Gas storage tank 14 has a volume of approximately 400 m³ and serves the known function of initial storage of the recovered gas.

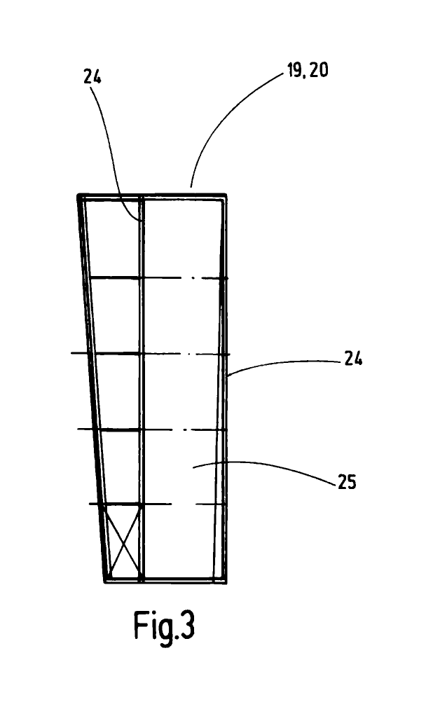

As is illustrated in FIG. 3, interior chamber 25 of a dry fermenter 19, 20 provided to contain the substrate has a rectangular cross-section. At the same time, the dry fermenter has a clear width of no more than 4.5 m and a clear height of no more than 5.0 m. Dry fermenters 19, 20 are also provided with laterally installed lattice segments 24 that are fitted with gutters. Lattice segments 24 are made from stainless steel and/or plastic and are inclined in such manner that their distance to the side walls of dry fermenters 19, 20 increases from bottom to top. In the embodiment of the invention shown in FIG. 3, the lattice segments are inclined at an angle of 2°.

The present invention is not limited in terms of its realisation to the preferred exemplary embodiment described in the preceding. Indeed, a number of variants are conceivable, even with fundamentally different arrangements, that are based on the solution presented. For example, the number of dry fermenters 19, 20 in a dry fermenter module 11, 12 may vary.

### KEY TO REFERENCE NUMBERS

100 Biomass power plant

10 Process water reservoir

11 Dry fermenter module

12 Dry fermenter module

13 Biofilter

14 Gas storage tank

15 Process water collecting duct

16 Staircase

17 Dry fermenter silo

18 Dry fermenter silo

19 Dry fermenter

20 Dry fermenter

21 Side

22 Side

23 Side

24 Lattice segment

25 Interior space

1. Biomass power plant for dry-wet simultaneous fermentation having dry fermenter modules comprising dry fermenters,
further including
a process water reservoir integrated between two dry fermenter modules (11, 12).

2. Biomass power plant as recited in claim 1,
wherein the dry fermenter modules each comprise four dry fermenters.

3. Biomass power plant as recited in claim 1,
wherein
process water conduits are constructed between the process water reservoir and both dry fermenter modules.

4. Biomass power plant as recited in claim 1,
wherein
the dry fermenters further comprise laterally installed lattice segments with gutters.

5. Biomass power plant as recited in claim 1,
wherein
the dry fermenters have a maximum clear width of 4.5 m and a maximum clear height of 5.0 m.

6. Biomass power plant as recited in claim 4,
wherein
the lattice segments are made from stainless steel and/or plastic.

7. Biomass power plant as recited in claim 4,
wherein
the lattice segments are inclined such that their distance to the side walls of the dry fermenters increases from bottom to top.

8. Biomass power plant as recited in claim 7,
wherein
the lattice segments are inclined by 2° in such manner that their distance to the side walls of the dry fermenters increases from bottom to top.

9. Biomass power plant as recited in claim 1,
wherein
the bottoms of the dry fermenters comprise isobaric air distribution devices.

10. Biomass power plant as recited in claim 1,
wherein
the dry fermenters are provided with nozzles integrated in the middle of the dry fermenter roof.

11. Biomass power plant as recited in claim 1,
wherein
the power plant includes a process water collecting duct that is coupled in the form of a module to the process water reservoir and/or the dry fermenters.

12. Biomass power plant as recited in claim 3,
wherein
the process water collecting duct has a volume of at least 50 m³.

13. Biomass power plant as recited in claim 11,
wherein
the process water collecting duct is arranged opposite a side of the process water reservoir not facing the dry fermenters.

14. Biomass power plant as recited in claim 1,
wherein
machine technology, piping, pumps, blowers and boosters arranged above the dry fermenter roof.

15. Biomass power plant as recited in claim 1,
wherein
a gas storage tank and/or a biofilter are arranged on the sides of the dry fermenter modules facing away from the process water reservoir.
